(19) United States
(12) Patent Application Publication
(10) Pub. No.: US 2011/0105364 A1
(43) Pub. Date: May 5, 2011

(54) COMPOSITIONS AND METHODS FOR TARGETED NUCLEIC ACID SEQUENCE SELECTION AND AMPLIFICATION

(75) Inventor: Nurith Kurn, Palo Alto, CA (US)

(73) Assignee: NuGEN Technologies, Inc., San Carlos, CA (US)

(21) Appl. No.: 12/938,112

(22) Filed: Nov. 2, 2010

Related U.S. Application Data
(60) Provisional application No. 61/257,241, filed on Nov. 2, 2009.

(51) Int. Cl. C40B 40/06; C40B 50/06; C12N 9/96

(52) U.S. Cl. 506/16; 506/26; 435/188

(57) ABSTRACT

The present invention provides novel methods, compositions, and kits for the production of amplification-ready, sequence-specific, target region-specific, and strand-specific regions of interest directly from samples containing complex DNA. The methods, composition, and kits provided herein are useful for selective target generation, genome partitioning, or user-selected enrichment of desired regions of interest. The invention described herein will enable multiplexing for genome-wide analysis with increased efficiency and is amenable to automation.

### CROSS REFERENCE

This application claims the benefit of U.S. Provisional Application No. 61/257,241 filed Nov. 2, 2009, which is herein incorporated by reference in its entirety.

### BACKGROUND OF THE INVENTION

The development of massively parallel sequencing technologies and platforms enable whole genome sequencing and targeted re-sequencing of defined organisms or mixtures of organisms. Although the cost of generating the newer sequencing information is decreasing and throughput of these technologies and platforms is increasing, it is recognized that focused target enrichment from high complexity genomic DNA will improve sequencing at high depth, enabling the sequencing or targeted re-sequencing of a larger number of samples as required for various fundamental biological studies of normal and disease development and pathogenesis.

Various methods for selective enrichment of a multiplicity of targets from genomic DNA, commonly referred to as “genome partitioning,” were developed in recent years. Some of these methods are based on selective hybridization to oligonucleotides designed to hybridize to the user-selected genomic regions. The hybridization can be to oligonucleotides immobilized on high or low density microarrays or solution phase hybridization to oligonucleotides modified with a ligand which can be subsequently immobilized to a solid surface, such as a bead. Other methods employ sequence-specific amplification (e.g., PCR) to amplify specific genomic regions in a droplet, allowing clonal amplification of defined regions for downstream sequencing.

A major drawback of the recently developed methods is the requirement to sequester regions of interest for hybridization to the specific oligonucleotides or to individual droplets. A related drawback to hybridization of surface-immobilized oligonucleotides is the low efficiency of the reaction, requiring large quantities of input DNA. These drawbacks lead to the need to fragment the original genomic DNA and in some instances further amplify the genomic DNA or library of fragments prior to selective enrichment by hybridization or sequestration to individual droplets

Therefore, there is a need for improved methods of selectively enriching target regions of genomic DNA for downstream next-generation applications such as massively parallel sequencing. The invention described herein fulfills this need.

### SUMMARY OF THE INVENTION

The present invention provides novel methods, compositions, and kits for the production of amplification-ready DNA of target sequence regions of interest partitioned from complex DNA, such as double stranded DNA, genomic DNA, or mixed DNA from more than one organism. The invention provides methods, compositions and kits for directly generating the DNA of target sequence regions of interest that can be used for selective target generation, genome partitioning, or user-selected enrichment of desired regions of interest. The invention described herein will enable multiplexing for genome-wide analysis with increased efficiency and is amenable to automation. The invention described herein will allow for selective enrichment of strand-specific sequence regions of interest, derived from a selected strand of highly complex double-stranded DNAs. The methods, compositions and kits further enable amplification of selective regions of interest, and specifically selective strands of interest by a variety of amplification methods, for example, the single primer isothermal linear amplification method (SPIA).

In one aspect, a method for selectively partitioning a plurality of target regions of interest from complex DNA is provided comprising a) hybridizing one or more oligonucleotides to the target regions of interest to form a plurality of target-oligonucleotide complexes; b) tethering a nucleic acid-modifying enzyme to a target-oligonucleotide complex via the oligonucleotide; and c) cleaving the target region of interest in the complex, thereby releasing the target region of interest from the enzyme.

In one embodiment, the one or more oligonucleotides hybridized to the target regions of interest are extended along the target region of interest by DNA polymerase prior to step b. In another embodiment, the complex DNA comprises double-stranded DNA. In another embodiment, the complex DNA comprises genomic DNA. In another embodiment, the genomic DNA comprises a mixture of genomic DNA from more than one organism. In another embodiment, the complex DNA comprises cDNA. In another embodiment, the cDNA is generated from a mixture of DNAs from more than one organism. In another embodiment, the selective partitioning is strand-specific. In another embodiment, the enzyme is a DNA duplex-specific endonuclease. In another embodiment, the enzyme is a restriction enzyme. In another embodiment, the method further comprises denaturing the complex DNA prior to hybridization of the oligonucleotides. In another embodiment, the method further comprises the formation of partial triplexes. In another embodiment, the method further comprises ligating adapters to the target regions of interest once released from the enzyme. In another embodiment, the adapter is selected from a group consisting of a double stranded adapter with an overhang at one end, a double stranded adapter with a 3′ single stranded overhang, an adapter that comprises a RNA-DNA heteroduplex, an adapter that comprises a chimeric DNA-RNA oligonucleotide and a stem-loop adapter. In another embodiment, the selective partitioning is carried out directly on the complex DNA. In another embodiment, said method does not involve amplifying the complex DNA prior to selective partitioning. In another embodiment, the method further comprises amplifying the partitioned target regions of interest thereby enriching for the target regions of interest. In another embodiment, the amplifying comprises single primer isothermal amplification. In another embodiment, the method further comprises sequencing of the amplified products. In another embodiment, the sequencing is performed using a massively parallel sequencing method. In another embodiment, the enzyme is synthetic, semisynthetic, or recombinant. In another embodiment, the ligand is selected from the group consisting of a small molecule, an antigen, an antibody, hybrid antibody or antibody fragment, an siRNA, an antisense RNA, an aptamer, a peptide and a first member of a specific binding pair. In another embodiment, the ligand-binding component is selected from the group consisting of a small molecule, an antigen, an antibody, hybrid antibody or antibody fragment, an siRNA, an antisense RNA, an aptamer, a protein therapeutic, or a peptide, a ligand binding protein, and a second member of specific binding pair. In another embodiment, at least 100 different regions of interest are selectively partitioned. In another embodiment, at least 100 oligonucleotides are used for selectively partitioning the target region of interest from complex DNA. In another embodiment, each oligonucleotide is coupled to the same ligand. In another embodiment, the ligand is biotin and the ligand-binding component is avidin or streptavidin.

In another aspect, a method of preparing amplification-ready selectively targeted regions of interest from double-stranded DNA is provided wherein the method comprises: a) denaturing the double-stranded DNA thereby generating single-stranded DNA; b) hybridizing to the single-stranded target regions of interest one or more oligonucleotides to form partial duplexes, each oligonucleotide being coupled to a ligand; c) contacting the ligands with a ligand-binding component coupled to a nucleic acid-modifying enzyme, wherein the enzyme cleaves the target regions of interest thereby obtaining products comprising the target regions of interest free of the enzyme; and d) ligating adapters to the products, whereby the targeted regions of interest are amplification-ready. In one embodiment, said method does not involve amplifying the double-stranded DNA until after step d.

In another aspect, a method of preparing amplification-ready selectively targeted regions of interest from double-stranded DNA is provided wherein the method comprises: a) hybridizing to the double-stranded target regions of interest one or more oligonucleotides to form partial triplexes, each oligonucleotide being coupled to a ligand; b) contacting the ligands with a ligand-binding component coupled to a nucleic acid-modifying enzyme, wherein the enzyme cleaves the target regions of interest thereby obtaining products comprising the target regions of interest free of the enzyme; and c) ligating adapters to the products, whereby the targeted regions of interest are amplification-ready. In one embodiment, said method does not involve amplifying the double-stranded DNA until after step c.

In one embodiment, the double-stranded DNA comprises genomic DNA. In another embodiment, the double-stranded DNA comprises cDNA. In another embodiment, the cDNA is generated from a mixture of DNAs from more than one organism. In another embodiment, the genomic DNA comprises a mixture of genomic DNA from more than one organism. In another embodiment, the method is strand-specific. In another embodiment, the enzyme is a DNA-duplex specific endonuclease. In another embodiment, the enzyme is a restriction enzyme. In another embodiment; the adapter is selected from a group consisting of a double stranded adapter with an overhang at one end, a double stranded adapter with a 3′ single stranded overhang, an adapter that comprises a RNA-DNA heteroduplex, an adapter that comprises a chimeric DNA-RNA oligonucleotide and a stem-loop adapter. In another embodiment, the method is carried out directly on the double-stranded DNA. In another embodiment, the method further comprises amplifying the target regions of interest, thereby enriching for the target sequence regions of interest. In another embodiment, amplifying comprises single primer isothermal amplification. In another embodiment, the method further comprises sequencing of the amplified products. In another embodiment, the sequencing is performed using a massively parallel sequencing method. In another method, the enzyme is synthetic, semisynthetic or recombinant. In another embodiment, the ligand is selected from the group consisting of a small molecule, an antigen, an antibody, hybrid antibody or antibody fragment, an siRNA, an antisense RNA, an aptamer, a peptide and a first member of a specific binding pair. In another embodiment, the ligand-binding component is selected from the group consisting of a small molecule, an antigen, an antibody, hybrid antibody or antibody fragment, an siRNA, an antisense RNA, an aptamer, a protein therapeutic, or a peptide, a ligand binding protein, and a second member of specific binding pair. In another embodiment, the ligand is biotin and the ligand-binding component is avidin or streptavidin. In another embodiment, at least 100 different regions of interest are prepared for amplification. In another embodiment, at least 100 oligonucleotides are used for preparing the target regions of interest for amplification. In another embodiment, each oligonucleotide is coupled to the same ligand.

In another aspect, a DNA complex is provided comprising: a) DNA comprising at least one DNA strand; b) an oligonucleotide hybridized to the DNA wherein the oligonucleotide is coupled to a ligand; and c) a ligand-binding component coupled to a nucleic acid-modifying enzyme, wherein the ligand and the ligand-biding component are further coupled to each other. In one embodiment, the DNA comprises genomic DNA. In another embodiment, the DNA is double stranded and the complex is a partial triplex. In another embodiment, the DNA is single stranded and the complex is a partial duplex. In another embodiment, the nucleic acid-modifying enzyme is synthetic, semisynthetic, or recombinant. In another embodiment, the ligand is selected from the group consisting of a small molecule, an antigen, an antibody, hybrid antibody or antibody fragment, an siRNA, an antisense RNA, an aptamer, a peptide and a first member of a specific binding pair. In another embodiment, the ligand binding component is selected from the group consisting of a small molecule, an antigen, an antibody, hybrid antibody or antibody fragment, an siRNA, an antisense RNA, an aptamer, a protein therapeutic, or a peptide, a ligand binding protein, and a second member of specific binding pair. In another embodiment, the ligand is biotin and the ligand binding component is avidin or streptavidin. In another embodiment, the oligonucleotide is further extended with a DNA polymerase.

In another aspect, a kit is provided comprising: a) one or more oligonucleotides, each coupled to a ligand; and b) a ligand-binding component that selectively binds to said ligand, wherein said ligand-binding component is coupled to a nucleic acid-modifying enzyme. In one embodiment, the kit further comprises reagents for amplification. In another embodiment, the kit further comprises an adapter for amplification. In another embodiment, the adapter is selected from a group consisting of a double stranded adapter with an overhang at one end, a double stranded adapter with a 3′ single stranded overhang, an adapter that comprises a RNA-DNA heteroduplex, an adapter that comprises a chimeric DNA-RNA oligonucleotide and a stem-loop adapter. In another embodiment, the kit further comprises reagents for sequencing. In another embodiment, the sequencing reagents comprise reagents for a massively parallel sequencing method. In another embodiment, the reagents are reagents for performing single primer isothermal amplification. In another embodiment, the kit further comprises a DNA polymerase. In another embodiment, the kit further comprises a plurality of oligonucleotides. In another embodiment, the plurality of oligonucleotides are coupled to different ligands. In another embodiment, the ligand is selected from the group consisting of a small molecule, an antigen, an antibody, hybrid antibody or antibody fragment, an siRNA, an antisense RNA, an aptamer, a peptide and a first member of a specific binding pair. In another embodiment, the ligand binding component is selected from the group consisting of a small molecule, an antigen, an antibody, hybrid antibody or antibody fragment, an siRNA, an antisense RNA, an aptamer, a protein therapeutic, or a peptide, a ligand binding protein, and a second member of specific binding pair. In another embodiment, the ligand is biotin and the ligand-binding component is avidin or streptavidin. In another embodiment, the nucleic acid modifying enzyme is synthetic, semisynthetic, or recombinant. In another embodiment, the nucleic acid modifying enzyme is a DNA duplex-specific endonuclease. In another embodiment, the nucleic acid modifying enzyme is a restriction endonuclease.

In another aspect, a plurality of amplification-ready partial DNA duplexes generated from genomic DNA is provided, each duplex comprising at least one DNA strand comprising the sequence of a target region of interest wherein the duplex is further ligated to an adaptor for single primer isothermal amplification.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts strand-specific selective hybridization.

FIG. 2 depicts selective tethering of a NA-modifying enzyme at target sequence regions of interest.

FIG. 3 depicts selective cleavage at target sequence regions of interest.

FIG. 4 depicts adapter ligation and selective amplification of target sequence regions of interest.

FIG. 5 depicts adapter ligation and selective amplification of target sequence regions of interest.

FIG. 6 depicts adapter ligation and selective amplification of target sequence regions of interest.

FIG. 7 depicts adapter ligation and selective amplification of target sequence regions of interest.

FIG. 8 depicts selective triplex formation and tethering of NA-modifying enzyme at target sequence regions of interest.

FIG. 9 depicts selective cleavage of dsDNA target sequence regions of interest.

### DETAILED DESCRIPTION OF THE INVENTION

### General Features

Selective enrichment of defined sequences from complex DNA according to the methods of the invention can be carried out in solution and can be based on the principle of tethering nucleic acid (NA) modifying enzymes to the specific regions of interest and subsequently generating amplification-ready products of the defined regions. The selection of regions of interest can be defined by the study objectives or any other user needs. Thus, for example, the regions of interest can represent all known coding regions, the entire exome, selected regions of coding genomic regions representing selected pathways, selected genomic regions known to comprise genomic variation related to altered phenotype, entire or selected regions of a specific chromosome, and the like.

The methods of the invention can be directed at selective, highly multiplex enrichment of genomic regions of interest from high complexity nucleic acid samples such as whole genomes or mixtures thereof. The selective enrichment can be carried out in solution comprising the high complexity nucleic acid samples and can employ sequence-specific hybridization, tethering of nucleic acid modifying (NA-modifying) enzyme (or enzymes) at the desired sequence of interest to enable generation of amplification-ready products of the defined sequence regions of interest. The methods can employ a multiplicity of oligonucleotides designed to hybridize to a multiplicity of target sequence regions of interest. The methods described herein can be used to obtain a plurality of different regions of interest from a complex nucleic acid sample containing complex DNA wherein the method can include incubating the nucleic acid sample with a plurality of different oligonucleotides, wherein the oligonucleotides selectively hybridize to each of the plurality of different regions of interest, and wherein each of the plurality of oligonucleotides can be indirectly coupled to a NA-modifying enzyme such as a DNA duplex-specific nuclease. The indirect coupling can be via binding between specific binding pairs such as a ligand and a ligand binding component.

Tethering the nucleic acid (NA)-modifying enzyme to the region(s) of interest can be achieved by hybridization of one or more oligonucleotides to the specific region(s) of interest. The target-specific oligonucleotide(s) can be modified with at least one ligand (referred to throughout interchangeably as a label or member of a specific-binding pair) which can serve as an anchor to bind a ligand binding component (for example, ligand binding protein, antibody, aptamer, or a second member of specific binding pair) conjugated to the NA-modifying enzyme.

The solution-based processes of the current invention can be more efficient than hybridization to immobilized oligonucleotides as commonly used in the recently developed genome partitioning methods. In addition to the efficient solution phase hybridization, the methods of the invention can employ various nucleic acid modifying enzymes as well as polymerases, which can be suitable for manipulation and amplification of very low input nucleic acid samples.

Insofar as the methods of the invention do not require fragmentation and sequestration of the input nucleic acid sample, the methods can be suitable to enrich sequence regions of interest from very low input nucleic acid samples. The invention described herein can be used to generate amplification-ready DNA of a target region of interest directly from a sample containing complex DNA without any prior manipulation of the sample such as fragmentation, amplification or sequestration.

Amplification-ready DNA of a target sequence region of interest can be generated directly from a sample containing complex DNA in a single reaction tube. In another embodiment, the methods described herein can be used to generate amplification-ready DNA of a target region of interest directly from a sample containing complex DNA in less than 20 minutes, 30 minutes, 60 minutes, 90 minutes, 120 minutes, 180 minutes, or 240 minutes, or 300 minutes. In another embodiment, the methods described herein can be used to generate amplification-ready DNA in about 20-300 minutes, 30-240 minutes, 60-180 minutes, 90 to 120 minutes, 20-240 minutes, 20-180 minutes, 20-120 minutes, 20-90 minutes, 20-60 minutes, or 20-30 minutes.

The methods described herein can be used to generate a collection of at least 25, 50, 75, 100, 500, 1000, 2500, 5000, 10,000, 25,000, 50,000, 100,000, 500,000, or 1,000,000 target sequence regions of interest directly from a sample of complex DNA using a plurality of oligonucleotides. The methods described herein can be used to generate a collection of about 1000 to 1,000,000, 2500 to 1,000,000, 5000 to 1,000,000, 10,000 to 1,000,000, 25,000 to 1,000,000, 50,000 to 1,000,000, 100,000 to 1,000,000, or 500,000 to 1,000,000 target sequence regions of interest directly from a sample of complex DNA using a plurality of oligonucleotides.

### Input Nucleic Acid Sample

The input is a nucleic acid. The input nucleic acid can be DNA, or complex DNA, for example genomic DNA. The input DNA may also be cDNA. The cDNA can be generated from RNA, e.g., mRNA. The input DNA can be of a specific species, for example, human, rat, mouse, other animals, specific plants, bacteria, algae, viruses, and the like. The input complex also can be from a mixture of genomes of different species such as host-pathogen, bacterial populations and the like. The input DNA can be cDNA made from a mixture of genomes of different species. Alternatively, the input nucleic acid can be from a synthetic source. The input DNA can be mitochondrial DNA. The input DNA can be cell-free DNA. The cell-free DNA can be obtained from, e.g., a serum or plasma sample. The input DNA can comprise one or more chromosomes. For example, if the input DNA is from a human, the DNA can comprise one or more of chromosome 1, 2, 3, 4, 5, 6, 7, 8, 9, 10, 11, 12, 13, 14, 15, 16, 17, 18, 19, 20, 21, 22, X, or Y. The DNA can be from a linear or circular genome. The DNA can be plasmid DNA, cosmid DNA, bacterial artificial chromosome (BAC), or yeast artificial chromosome (YAC). The input DNA can be from more than one individual or organism. The input DNA can be double stranded or single stranded. The input DNA can be part of chromatin. The input DNA can be associated with histones.

In so far as the methods of the invention do not require fragmentation and sequestration of the input nucleic acid sample, the methods can be suitable for enrichment of desired sequence regions of interest from very low input nucleic acid samples. In certain embodiments, input as low as a picogram or a nanogram of nucleic acid are sufficient. The methods of the current invention can be suitable for enrichment of selected sequence regions of interest from complex genomic nucleic acid samples. The amount of input nucleic acid can be, e.g., less than 1 pg, 10 pg, 100 pg, 1 ng, 10 ng, 100 ng, 1 μg, 10 μg, or 100 μg. The amount of input nucleic acid can be, e.g., about 1 pg to 1 ng, 1 pg to 1 μg; or 1 ng to 1 μg.

Insofar as no prior denaturation of the input nucleic acid sample is required, the method of the invention can be suitable for in situ selective enrichment and amplification of sequence regions of interest.

In some embodiments, the oligonucleotides targeting the selected sequence regions of interest are designed to hybridize to ssDNA targets. In the case where the input nucleic acid sample comprises genomic DNA or other dsDNA, the input nucleic acid sample can be first denatured to render the target single stranded and enable hybridization of the oligonucleotides to the desired sequence regions of interest. In these embodiments, the methods and compositions described herein can allow for region-specific enrichment and amplification of sequence regions of interest.

In other embodiments, the oligonucleotides targeting the selected sequence regions of interest are designed to hybridize to dsDNA target, without denaturation of the dsDNA. In these embodiments, the oligonucleotides targeting the selected sequence regions of interest are designed to form a triple helix (triplex) at the selected sequence regions of interest. The hybridization of the oligonucleotides to the dsDNA sequence regions of interest can be carried out without prior denaturation of the double stranded nucleic acid sample. In such embodiments, the methods and compositions described herein can allow for region-specific enrichment as well as strand-specific enrichment and amplification of sequence regions of interest. This method can be useful for generation of copies of strand specific sequence regions of interest from complex nucleic acid without the need to denature the dsDNA input DNA, thus enabling enrichment and analysis of multiplicity of sequence regions of interest in the native complex nucleic acid sample. The method can find use for studies and analyses carried out in situ, enable studies and analysis of complex genomic DNA in single cells or collection of very small well defined cell population, as well as permit the analysis of complex genomic DNA without disruption of chromatin structures.

### Oligonucleotides

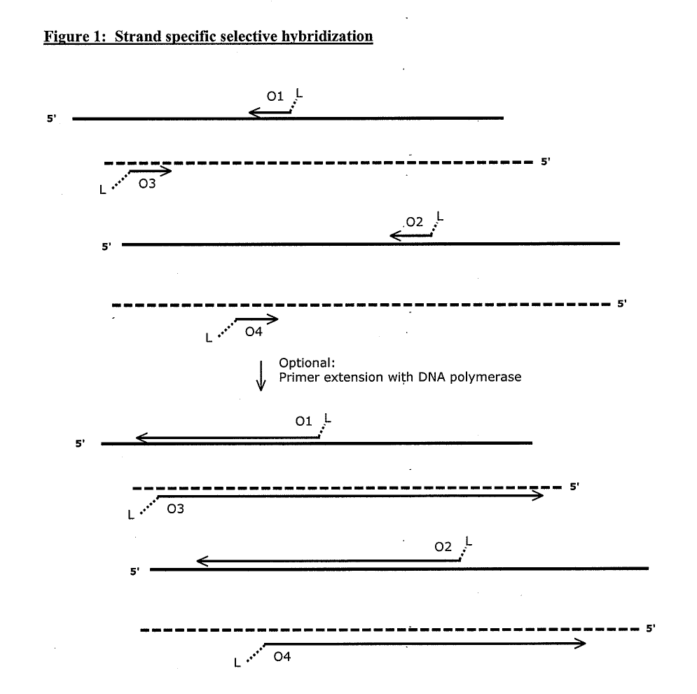

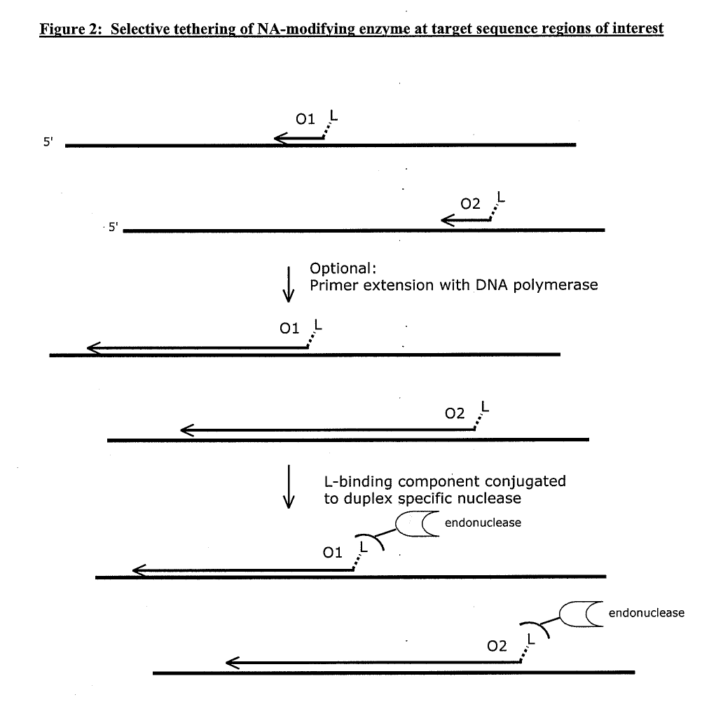

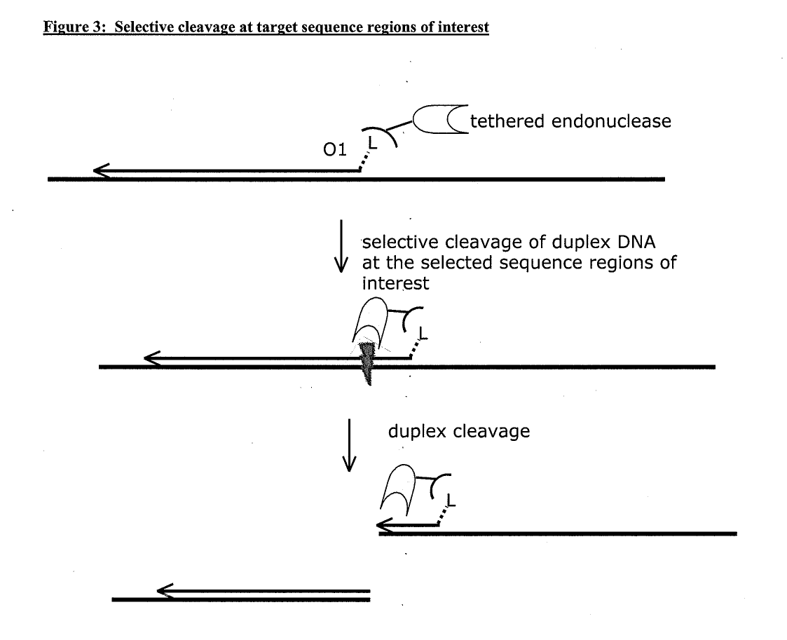

In one embodiment of the invention, the oligonucleotides of the invention hybridize to single stranded targets to form duplexes, as is exemplified in FIG. 1. This hybridization is then followed by tethering a NA-modifying enzyme at the sequence region of interest and selective cleavage of dsDNA sequence region of interest, as is depicted in FIGS. 2-3.

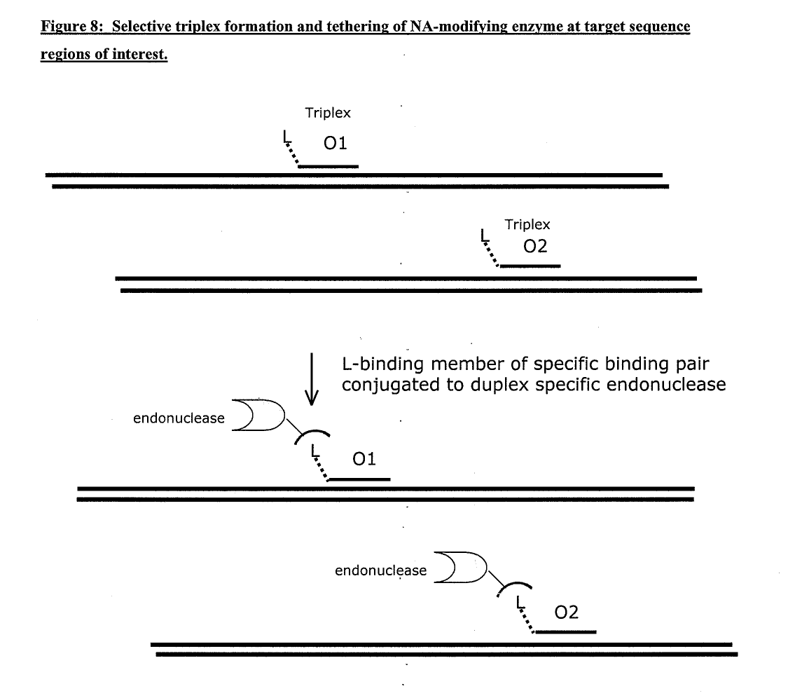

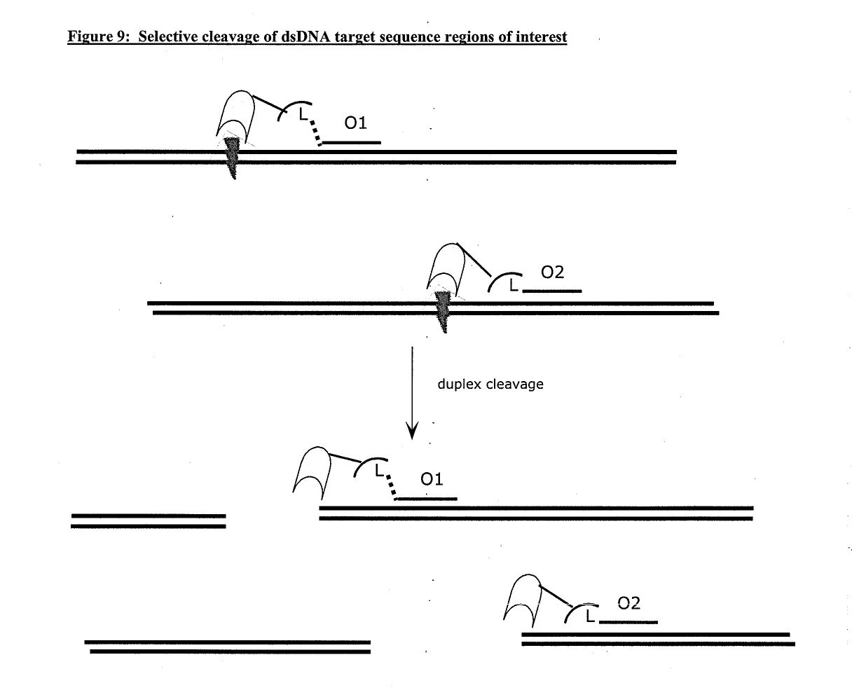

In another embodiment of the invention the oligonucleotides of the invention hybridize to double stranded targets to form a triplex nucleic acid, as is exemplified in FIG. 8. In this embodiment, this hybridization is then followed by tethering of a NA-modifying enzyme at the sequence region of interest and selective cleavage of a dsDNA sequence region of interest, as is depicted in FIGS. 8-9.

In some embodiment of the invention, the hybridization of the oligonucleotide or oligonucleotides to the target dsDNA can be carried out without prior denaturation by recombinase mediated hybridization, for example RecA mediated specific hybridization, for example as described in U.S. Pat. Nos. 5,965,361 and 7,468,244 and references therein. The best known RecA-like recombinase is the RecA protein from E. coli, which is available commercially (Roche Applied Science, Indianapolis, hid. USA).

RecA orthologues have also been identified in a wide variety of prokaryotic genera, including Bacillus (B. halodurans, GenBank accession no. NP_{—}243249), Streptomyces (S. agalactiae, GenBank accession no. NP_{—}689079; S. pyogenes MGAS315, GenBank accession no. NP_{—}665604); Staphylococcus (S. aureus subsp. Aureus MW2, GenBank accession no. NP_{—}645985); Brucella (B. melitensis, GenBank accession no. NP_{—}539704); Helicobacter (H. pylori, GenBank accession no. NP_{—}206952); Corynebacterium (C. glutamicum, GenBank accession no. NP_{—}601162); Bordetella (B. hinzii, GenBank accession no. AAM92267); Bacteroides (B. fragilis, GenBank accession no. AAK58827); Haemophilus (H. influenzae, GenBank accession no. AAM91954); archaebacteria (Reich et al., Extremophiles 5(4):265-75 (2001); and others.

In eukaryotes, RecA orthologues are typically members of the Rad51 family (Shibata et al., Proc. Natl. Acad. Sci. USA 98:8425-8432 (2001)), members of which have been identified in a wide variety of eukaryotic organisms including yeasts, such as Saccharomyces cerevisiae (GenBank accession no. NP_{—}011021), Drosophila melanogaster (GenBank accession no. Q27297), Caenorhabditis elegans (GenBank accession no. BAA24982), and homo sapiens (Yoshimura et al., “Cloning and sequence of the human RecA-like gene cDNA,” Nucl. Acids Res. 21(7):1665 (1993); GenBank accession no. NP_{—}002866).

Allelic variants and engineered mutants (collectively, muteins) of RecA and of its orthologues have also been described, including recA-803 (Madiraju et al., Proc. Natl. Acad. Sci. USA 85(18):6592-6 (1988).

In some embodiments, multiple oligonucleotides are employed to hybridize to a multiplicity of genomic regions of interest. The multiplicity of oligonucleotides can be directed to hybridize to a large genomic region of interest, allowing selective enrichment of a large genome region. The multiplicity of oligonucleotides can be directed to hybridize to a large genomic region of interest, allowing selective enrichment of a large genome region directed to hybridize to a multiplicity of genomic regions of interest on a single chromosome or multiplicity of chromosomes. The chromosomes can be of a specific species (for example, human, rat, mouse, other animals, specific plants; bacteria, algae, viruses, and the like) or a mixture of chromosomes of different species (such as host-pathogen, bacterial populations, and the like). Insofar as the genomic regions of interest may be very long, multiple oligonucleotides can be designed to hybridize to different sequence regions within the genomic region of interest. Tiling of oligonucleotides specific for hybridization across targeted genomic regions of interest is also envisioned.

The length of the oligonucleotides designed to carry out the methods of the invention can depend on the application, or user-defined criteria, and can be determined for maximum specificity. In addition, when a multiplicity of oligonucleotides are employed, the sequence/composition (e.g., % GC content) and length of the oligonucleotides can be designed to ensure specific and efficient hybridization at the same stringency for hybridization to all genomic regions of interest, thus enabling highly multiplex target enrichment process. It is clear to persons skilled in the art that the sequence content and oligonucleotide length can be adjusted to provide efficient and specific hybridization to the defined genomic regions of interest under a defined stringency as determined by the solution composition and temperature.

The oligonucleotides of the invention can be synthetic polynucleotides that are single stranded and contain a sequence that is capable of hybridizing with a sequence of the target input DNA. The region of an oligonucleotide that hybridizes with the target nucleic acid can be at least 80%, 90%, 95%, or 100% complementary to the target sequence. The number of nucleotides in the hybridizable sequence of a specific oligonucleotide can be such that stringency conditions used to hybridize the oligonucleotide can prevent excessive random non-specific hybridization. Usually, the number of nucleotides in the hybridizing portion of an oligonucleotide can be at least as great as the defined sequence on the target polynucleotide that the oligonucleotide hybridizes to, namely, at least 10, at least 15, at least 20, at least 25, or at least 30 nucleotides, or generally from about 25 to about 100 nucleotides, or from about 25 to about 60 nucleotides, or from about 25 to about 50 nucleotides, or from about 25 to about 35 nucleotides, or from about 20 to about 30 nucleotides. The melting temperature (Tₘ) of oligonucleotides with different sequences in a reaction can be similar or different.

Large sets of oligonucleotides can be synthesized by various methods. Methods for highly parallel synthesis of a large number of oligonucleotides on microarrays or other solid surface configuration (e.g., micro fluidic array platform) were developed in recent years (for example, by Affymetrix, Nimblegen, Agilent, Febit, LC Science and others). The number of oligonucleotides with different sequences that can be used in the methods of provided invention can be at least 10, 25, 50, 75, 100, 250, 500, 750, 1000, 2500, 5000, 7500, 10,000, 25,000, 50,000, 75,000, 100,000, 250,000, 500,000, 750,000, or 1,000,000. The number of oligonucleotides with different sequences that can be used in the methods of the provided invention can be about 100-1000, about 1000-10,000, about 10,000 to 100,000, or about 100,000 to 1,000,000.

In some embodiments, the hybridized oligonucleotides are extended along the sequence region of interest by a DNA polymerase.

### Ligands, Ligand Binding Components, and Specific Binding Pairs

The oligonucleotides used for carrying out the methods of the invention can comprise at least one label or ligand at one end. In some embodiments the label or ligand is attached to the oligonucleotides at the 5′-end. In other embodiments, the oligonucleotides have a label or ligand attached at both 3′- and 5′-ends. In another embodiment, the label or ligand is attached to only the 3′ end of the oligonucleotide. Each end may be modified with different labels or ligands, enabling targeted tethering of defined NA-modifying enzymes to each end. Alternatively, the labels or ligands at each end of an oligonucleotide can be the same.

Generally, a ligand can be any compound for which a ligand-binding component or receptor naturally exists or can be prepared. A ligand can be a compound that is designed and engineered to interact with a receptor. Similarly, a ligand-binding component or receptor or anti-ligand can be generally any compound or composition capable of recognizing a particular spatial and polar organization of a ligand molecule, e.g., epitopic or determinant site.

The ligand and ligand-binding component can be considered to be members of a specific binding pair. Generally a specific binding pair member can be one of two different molecules (either the ligand or the ligand-binding component) having an area on the surface or in a cavity which specifically binds to and is thereby defined as complementary with a particular spatial and polar organization of the other molecule. The members of the specific binding pair can be referred to as a ligand and ligand-binding component. Exemplary embodiments include an immunological pair such as antigen-antibody, operator-repressor, nuclease-nucleotide, biotin-avidin, hormone-hormone receptor, IgG-protein A, DNA-DNA, DNA-RNA, and the like. Additional exemplary embodiments of ligand and ligand-binding components include ligand-receptor, substrate-enzyme (e.g., substrate-kinase, substrate-protease, substrate-phosphatase), protein domain homo-oligomers (e.g., homo-dimers, homo-trimers, homo-tetramers, or homo-polymers), protein domain hetero-oligomers (e.g., hetero-dimers, hetero-trimers, hetero-tetramers, or hetero-polymers), nucleic acid-protein nucleic acid binding domain (e.g., DNA-protein DNA binding domain, RNA-protein RNA binding domain), phosphate containing molecule-phospho binding protein, methylation containing molecule-methylation binding protein, acetyl-lysine containing molecule-bromodomain, phospho-Y-containing peptide-SH2 domain, calmodulin binding peptide (CBP)-calmodulin, or histidine tag-nickel, etc. The specific binding pair can be a pair of proteins that interact as found in the Database of Interacting Proteins (DIP) (http://dip.doe-mbi.ucla.edu/dip/Main.cgi) or the Database of Ligand-Receptor Partners (DLRP) (http://dip.doe-mbi.ucla.edu/dip/DLRP.cgi). An interacting partner can be a protein interaction domain described at http://pawsonlab.mshri.on.ca/index.php?option=com_content&task=view&id=30&Itemid=63.

In exemplary embodiments a ligand can be selected from but not limited to a small molecule, an antigen, an antibody, hybrid antibody or antibody fragment, an siRNA, an antisense RNA, an aptamer, a polypeptide, a peptide, and a first member of a specific binding pair such as biotin or avidin/streptavidin.

In exemplary embodiments a ligand binding component can be selected from but not limited to a ligand binding protein, a small molecule, an antigen, an antibody, hybrid antibody or antibody fragment, an siRNA, an antisense RNA, an aptamer, a protein, a polypeptide, a peptide, and a second member of a specific binding pair such as avidin/streptavidin or biotin. Related exemplary ligand-binding components include naturally occurring and synthetic receptors, antibodies, enzymes, Fab fragments, lectins, nucleic acids, repressors, oligonucleotides, protein A, complement component C1q, or DNA binding proteins and the like.

### Nucleic Acid Modifying Enzymes

The NA-modifying enzyme can be DNA-specific modifying enzyme. The NA-modifying enzyme can be a duplex-specific modifying enzyme.

The NA-modifying enzyme can be selected for specificity for dsDNA. The enzyme can be a duplex-specific endonuclease, a blunt-end frequent cutter restriction enzyme, or other restriction enzyme. Examples of blunt-end cutters include DraI or SmaI. The NA-modifying enzyme can be an enzyme provided by New England Biolabs (www.neb.com). The NA-modifying enzyme can be a homing endonuclease (a homing endonuclease can be an endonuclease that does not have a stringently-defined recognition sequence). The NA-modifying enzyme can be a nicking endonuclease (a nicking endonuclease can be an endonuclease that can cleave only one strand of DNA in a double-stranded DNA substrate). The NA-modifying enzyme can be a high fidelity endonuclease (a high fidelity endonuclease can be an engineered endonuclease that has less “star activity” than the wild-type version of the endonuclease). The NA-modifying enzyme can be an endonuclease that has a methylated recognition site (e.g., DpnI). The NA-modifying enzyme can be a 3-base cutter, 4-base cutter, 5-base cutter, 6-base cutter, 7-base cutter, 8-base cutter, 9-base cutter, 10-base cutter, 11-base cutter, 12-base cutter, 13-base cutter, 14-base cutter, 15-base cutter, 16-base cutter, 17-base cutter, 18-base cutter, 19-base cutter, or 20-base cutter endonuclease. The NA-modifying enzyme can be a thermophilic restriction enzyme. A restriction enzyme can recognize a palindromic sequence.

The NA-modifying enzyme can be a Type I endonuclease, a Type II endonuclease, a Type III endonuclease, or endoribonuclease. The NA-modifying enzyme can be a structure specific nuclease (e.g., a flap endonuclease (FEN)), a sequence specific nuclease, an endonuclease that can generate a “blunt end,” an endonuclease that can generate a 1, 2, 3, 4, 5, 6, 7, 8, 9, or 10 base 5′ overhang, an endonuclease that can generate a 1, 2, 3, 4, 5, 6, 7, 8, 9, or 10 base 3′ overhang, a micrococcal nuclease (e.g., S7 Nuclease or MNase), mung bean nuclease, an S1 nuclease, nuclease Bal-31, or a P1 nuclease.

The NA-modifying enzyme can be an exonuclease (e.g., 3′ to 5′ exonuclease (e.g., exonuclease I, exonuclease III, exonuclease T), a 5′ to 3′ exonuclease (e.g., Lambda exonuclease, T7 exonuclease), a poly(A)-specific 3′ to 5′ exonuclease, a 3′ to 5′ exoribonuclease), a polymerase (e.g., a DNA polymerase, an RNA polymerase), a ligase (e.g., a DNA ligase), a phosphatase (e.g., a DNA phosphatase, a 3′ phosphatase, alkaline phosphatase), a kinase (e.g., a DNA kinase, e.g., T4 polynucleotide kinase), a DNA methyltransferase, DNA gyrase, uracil-DNA glycosylase (UDG), a topoisomerase, a recombinase (e.g., Cre Recombinase), or a helicase.

Other NA modification enzymes are also envisioned to be useful for carrying out the methods of the invention.

The NA-modifying enzyme employed can require interaction with solution phase subunit. Various duplex-specific restriction enzymes require dimer formation for cleavage of both strands of a duplex. This required dimerization (the interaction of the tethered enzyme and a solution phase enzyme) can increase the specificity of cleavage by limiting it to the site of the sequence regions of interest.

The NA-modifying enzymes can be natural, recombinant, synthetic, or semi-synthetic. The NA-modifying enzymes can be chemical enzyme mimics. For example, such enzymes can be isolated from specific organisms or recombinantly generated. Exemplary NA-modifying enzymes include but are not limited to the duplex-specific nuclease from the hepatopancreas of the Kamchatka crab, Drosophila zinc-finger nucleases, and targeting individual subunits of the FokI restriction endonuclease to specific DNA strands. The action of the NA-modifying enzyme at selective sites in the sequence regions of interest can result in marking the sites for subsequent reaction, or lead to dsDNA cleavage which can render the sites suitable for appending desired adapters for a variety of downstream applications such as amplification, library generation, and sequencing.

### Linkers

The label or ligand can be linked to the oligonucleotide by a linker, the synthesis of which is known in the art. The length and flexibility of the linker can affect the flexibility and reach of the tethered NA-modifying enzyme, as will be explained in the following. Various linkers are known in the art and are useful for carrying out the methods of the invention. The linker can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40 or more covalent bonds. The linker can comprise at least 1, 2, 3, 4, 5, 6, 7, 8, 9, 10, 11, 12, 13, 14, 15, 16, 17, 18, 19, 20 or more carbons.

The flexibility and length of linkers used in the method of the invention can be designed to influence the reach of the NA-modifying enzyme to act on the desired sequence region of interest, or act on proximal sequences which can be within reach. Thus, linkers of defined flexibility and length can be used for attaching the label or ligand to the oligonucleotides, as well as the linkers used to attach the NA-modifying enzyme to the member of the specific binding pair (for binding to the label or ligand).

The attachment of the NA-modifying enzyme to the ligand member of the specific binding pair can be engineered so that the attachment is directed to specific moieties of either or both the member of the specific binding pair and the NA-modifying enzyme. Furthermore, the attachment can be genetically engineered to incorporate the desired moieties into either the member of the specific binding pair or the NA-modifying enzyme or both. A genetically engineered fused product of the member of the specific binding pair and the NA-modifying enzyme is also envisioned. Flexible linkers are well known in the art, for example PEG (polyethylene glycol) or PEO (hydrophilic polyethylene oxide) spacer arms of various lengths (for example as provided by Thermo Scientific (http://www.piercenet.com/products/browse.cfm?fldID=062CC432-FBF8-4E9D-8930-458385BCED1D).

### Adapters

Various adapter designs are envisioned which are suitable for generation of amplification-ready products of target sequence regions/strands of interest. Ligation of adapters at the desired end of the sequence regions of interest is suitable for carrying out the methods of the invention. Various ligation modalities are envisioned, dependent on the choice of NA-modifying enzymes and the resulting dsDNA cleavage. For example, when a blunt end product comprising the target region/sequence of interest is generated, blunt end ligation can be suitable. Alternatively, where the cleavage is carried out using a restriction enzyme of known sequence specificity, leading to the generation of cleavage sites with known sequence overhangs, suitable ends of the adapters can be designed to enable hybridization of the adapter to the cleavage site of the sequence region of interest and subsequent ligation. Reagents and methods for efficient and rapid ligation of adapters are commercially available and are known in the art.

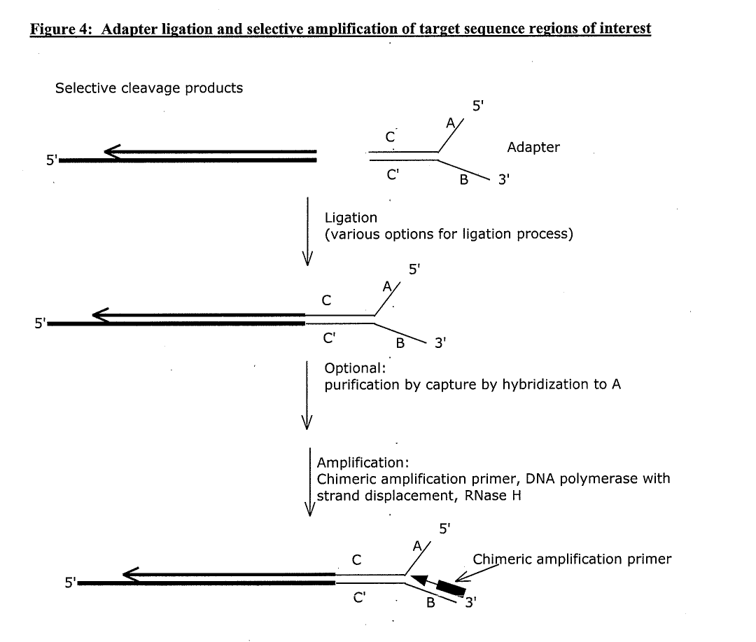

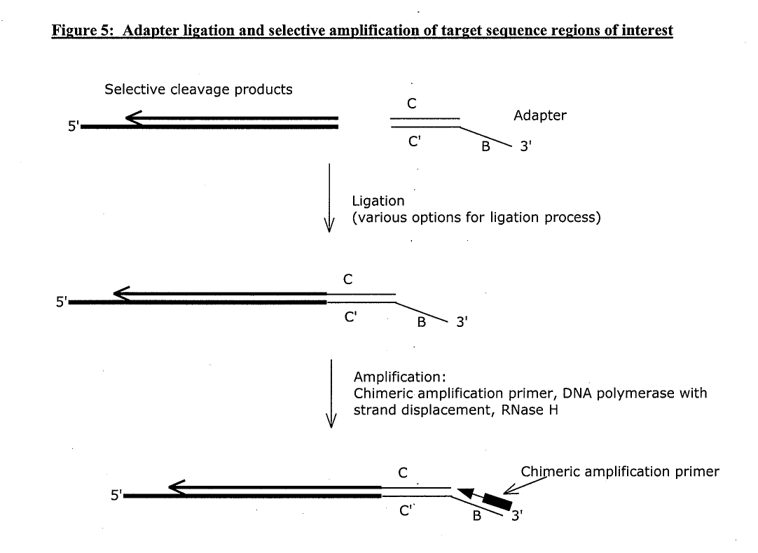

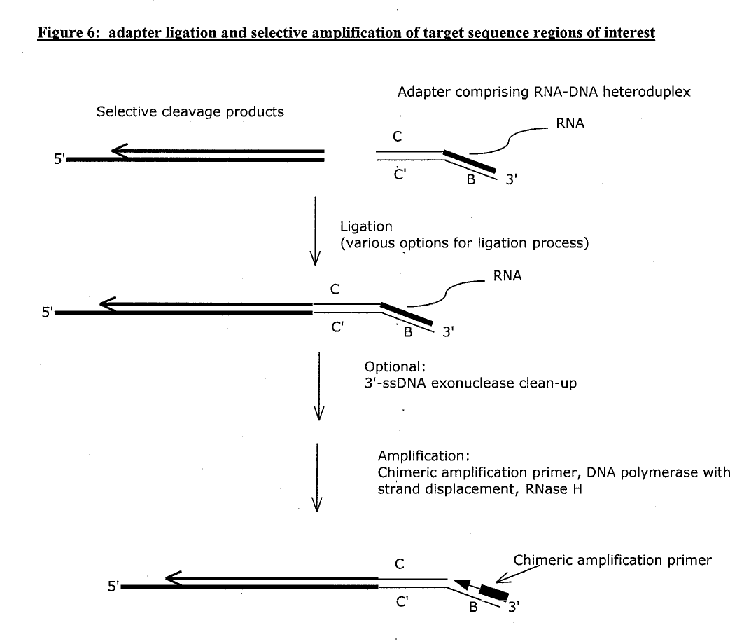

Various adapter designs are envisioned. Exemplary adapter designs are shown in FIGS. 4-7.

The adapters can be designed to comprise a double-stranded portion which can be ligated to the dsDNA (or dsDNA with overhang) products. Various ligation processes and reagents are known in the art and can be useful for carrying out the methods of the invention. For example, blunt ligation can be employed. Similarly, a single dA nucleotide can be added to the 3′-end of the dsDNA product, by a polymerase lacking 3′-exonuclease activity and can anneal to an adapter comprising a dT overhang (or the reverse). This design allows the hybridized components to be subsequently ligated (e.g., by T4 DNA ligase). Other ligation strategies and the corresponding reagents and known in the art and kits and reagents for carrying out efficient ligation reactions are commercially available (e.g, from New England Biolabs, Roche).

A common feature of the adapter designs depicted in FIGS. 4 to 7 is the 3′-single stranded DNA comprising a unique sequence (the sequence of an amplification primer) which can hybridize to an amplification primer employed in a subsequent amplification step, thus rendering the ligation product suitable for subsequent amplification of the sequence regions of interest.

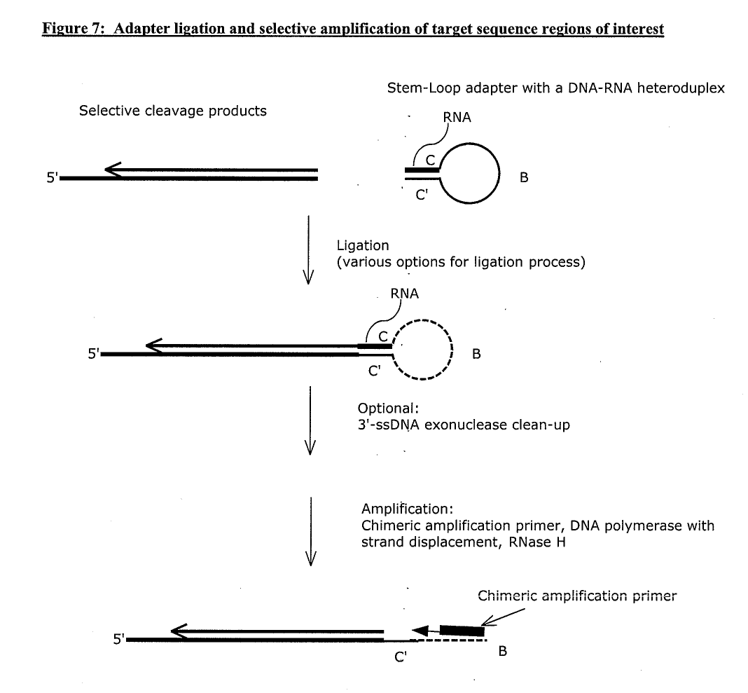

A novel stem-loop adapter design is shown in FIG. 7. The stem portion of the adapter comprises an RNA-DNA heteroduplex formed by hybridization of the 3′-portion of the oligonucleotide, comprising RNA sequence, to its 5′-end portion. The 3′-end of the oligonucleotide can comprise a dNTP to facilitate ligation when required for efficient ligation using the ligase of choice. The stem may or may not be blunt ended, as required for efficient hybridization to the cleavage products of the sequence sequences of interest generated by the methods of the invention. In this embodiment, the ligation products comprise a loop at one end attached to the cleavage product via an RNA-DNA heteroduplex. The RNA portion of the heteroduplex is susceptible to cleavage by RNase H. The adapter further comprises a loop sequence that is hybridizable to an amplification primer used for the single primer amplification of the sequence regions of interest. Cleavage of the RNA portion of the stem by RNase H results is the formation of ssDNA 3′-overhang suitable which is the primer binding site for downstream amplification.

The dsDNA portion of the adapters can further comprise indexing or bar-coding sequences designed to mark either the samples or sequences of interest. The ability to incorporate marking and/or bar-coding information in the adapter design can further expand the high multiplexing ability of the methods of the invention.

### Methods of Amplification

The methods, compositions and kits described herein can be useful to generate amplification-ready products directly from genomic DNA for downstream applications such as massively parallel sequencing (Next Generation Sequencing methods), multiplexed quantification of large sets of sequence regions of interest, such as by high density qPCR arrays and other highly parallel quantification platforms (selective massively parallel target pre-amplification), as well as generation of libraries with enriched population of sequence regions of interest. The methods described herein can be used to generate a collection of at least 25, 50, 75, 100, 500, 1000, 2500, 5000, 10,000, 25,000, 50,000, 100,000, 500,000, or 1,000,000 amplification-ready target sequence regions of interest directly from a sample of complex DNA using a plurality of oligonucleotides.

Methods of nucleic acid amplification are well known in the art. In some embodiments, the amplification method is isothermal. In other embodiments the amplification method is linear. In other embodiments the amplification is exponential.

SPIA Amplification

Amplification of the sequence regions of interest employing a linear amplification method such as the single primer isothermal amplification (SPIA) can be used. SPIA enables generation of multiple copies of the strand specific sequence regions of interest and employs a single amplification primer, thus reducing the complexity associated with multiple oligonucleotide design and manufacturing, enables the use of a generic amplification primer, and is linear. The fidelity of quantification of the copy number of the sequence regions of interest in the complex genomic NA sample is a highly desirable feature of the presented methods of the invention.

Amplification by SPIA can occur under conditions permitting composite primer hybridization, primer extension by a DNA polymerase with strand displacement activity, cleavage of RNA from a RNA/DNA heteroduplex and strand displacement. In so far as the composite amplification primer hybridizes to the 3′-single-stranded portion (of the partially double stranded polynucleotide which is formed by cleaving RNA in the complex comprising a RNA/DNA partial heteroduplex) comprising, generally, the complement of at least a portion of the composite amplification primer sequence, composite primer hybridization may be under conditions permitting specific hybridization. In SPIA, all steps are isothermal (in the sense that thermal cycling is not required), although the temperatures for each of the steps may or may not be the same. It is understood that various other embodiments can be practiced given the general description provided above. For example, as described and exemplified herein, certain steps may be performed as temperature is Changed (e.g., raised, or lowered).

Although generally only one composite amplification primer is described above, it is further understood that the SPIA amplification methods can be performed in the presence of two or more different first and/or second composite primers that randomly prime template polynucleotide. In addition, the amplification polynucleotide products of two or more separate amplification reactions conducted using two or more different first and/or second composite primers that randomly prime template polynucleotide can be combined.

The composite amplification primers are primers that are composed of RNA and DNA portions. In the amplification composite primer, both the RNA and the DNA portions are generally complementary and can hybridize to a sequence in the amplification-ready product to be copied or amplified. In some embodiments, a 3′-portion of the amplification composite primer is DNA and a 5′-portion of the composite amplification primer is RNA. The composite amplification primer is designed such that the primer is extended from the 3′-DNA portion to create a primer extension product. The 5′-RNA portion of this primer extension product in a RNA/DNA heteroduplex is susceptible to cleavage by RNase H, thus freeing a portion of the polynucleotide to the hybridization of an additional composite amplification primer. The extension of the amplification composite primer by a DNA polymerase with strand displacement activity releases the primer extension product from the original primer and creates another copy of the sequence of the polynucleotide. Repeated rounds of primer hybridization, primer extension with strand displacement DNA synthesis, and RNA cleavage create multiple copies of the strand-specific sequence of the polynucleotide.

Other Amplification Methods

Some aspects of the invention comprise the amplification of polynucleotide molecules or sequences within the polynucleotide molecules. Amplification generally refers to a method that can result in the formation of one or more copies of a nucleic acid or polynucleotide molecule or in the formation of one or more copies of the complement of a nucleic acid or polynucleotide molecule. Amplifications can be used in the invention, for example, to amplify or analyze a polynucleotide bound to a solid surface. The amplifications can be performed, for example, after archiving the samples in order to analyze the archived polynucleotide.

In some aspects of the invention, exponential amplification of nucleic acids or polynucleotides is used. These methods often depend on the product catalyzed formation of multiple copies of a nucleic acid or polynucleotide molecule or its complement. The amplification products are sometimes referred to as “amplicons.” One such method for the enzymatic amplification of specific double stranded sequences of DNA is polymerase chain reaction (PCR). This in vitro amplification procedure is based on repeated cycles of denaturation, oligonucleotide primer annealing, and primer extension by thermophilic template dependent polynucleotide polymerase, resulting in the exponential increase in copies of the desired sequence of the polynucleotide analyte flanked by the primers. The two different PCR primers, which anneal to opposite strands of the DNA, are positioned so that the polymerase catalyzed extension product of one primer can serve as a template strand for the other, leading to the accumulation of a discrete double stranded fragment whose length is defined by the distance between the 5′ ends of the oligonucleotide primers. Other amplification techniques that can be used in the methods of the provided invention include, e.g., AFLP (amplified fragment length polymorphism) PCR (see e.g: Vos et al. 1995. AFLP: a new technique for DNA fingerprinting. Nucleic Acids Research 23: 4407-14), allele-specific PCR (see e.g., Saiki R K, Bugawan T L, Horn G T, Mullis K B, Erlich H A (1986). Analysis of enzymatically amplified beta-globin and HLA-DQ alpha DNA with allele-specific oligonucleotide probes Nature 324: 163-166), Alu PCR, assembly PCR (see e.g., Stemmer W P, Crameri A, Ha K D, Brennan T M, Heyneker H L (1995). Single-step assembly of a gene and entire plasmid from large numbers of oligodeoxyribonucleotides Gene 164: 49-53), assymetric PCR (see e.g., Saiki R K supra), colony PCR, helicase dependent PCR (see e.g., Myriam Vincent, Yan Xu and Huimin Kong (2004). Helicase-dependent isothermal DNA amplification EMBO reports 5 (8): 795-800), hot start PCR, inverse PCR (see e.g., Ochman H, Gerber A S, Hartl D L. Genetics. 1988 November; 120(3):621-3), in situ PCR, intersequence-specific PCR or IS SR PCR, digital PCR, linear-after-the-exponential-PCR or Late PCR (see e.g., Pierce K E and Wangh L T (2007). Linear-after-the-exponential polymerase chain reaction and allied technologies Real-time detection strategies for rapid, reliable diagnosis from single cells Methods Mol. Med. 132: 65-85), long PCR, nested PCR, real-time PCR, duplex PCR, multiplex PCR, quantitative PCR, or single cell PCR.

Another method for amplification involves amplification of a single stranded polynucleotide using a single oligonucleotide primer. The single stranded polynucleotide that is to be amplified contains two non-contiguous sequences that are complementary to one another and, thus, are capable of hybridizing together to form a stem-loop structure. This single stranded polynucleotide already may be part of a polynucleotide analyte or may be created as the result of the presence of a polynucleotide analyte.

Another method for achieving the result of an amplification of nucleic acids is known as the ligase chain reaction (LCR). This method uses a ligase enzyme to join pairs of preformed nucleic acid probes. The probes hybridize with each complementary strand of the nucleic acid analyte, if present, and ligase is employed to bind each pair of probes together resulting in two templates that can serve in the next cycle to reiterate the particular nucleic acid sequence.

Another method for achieving nucleic acid amplification is the nucleic acid sequence based amplification (NASBA). This method is a promoter-directed, enzymatic process that induces in vitro continuous, homogeneous and isothermal amplification of a specific nucleic acid to provide RNA copies of the nucleic acid. The reagents for conducting NASBA include a first DNA primer with a 5′-tail comprising a promoter, a second DNA primer, reverse transcriptase, RNAse-H, T7 RNA polymerase, NTP's and dNTP's.

Another method for amplifying a specific group of nucleic acids is the Q-beta-replicase method, which relies on the ability of Q-beta-replicase to amplify its RNA substrate exponentially. The reagents for conducting such an amplification include “midi-variant RNA” (amplifiable hybridization probe), NTP's, and Q-beta-replicase.

Another method for amplifying nucleic acids is known as 3SR and is similar to NASBA except that the RNAse-H activity is present in the reverse transcriptase. Amplification by 3SR is an RNA specific target method whereby RNA is amplified in an isothermal process combining promoter directed RNA polymerase, reverse transcriptase and RNase H with target RNA. See for example Fahy et al. PCR Methods Appl. 1:25-33 (1991).

Another method for amplifying nucleic acids is the Transcription Mediated Amplification (TMA) used by Gen-Probe. The method is similar to NASBA in utilizing two enzymes in a self-sustained sequence replication. See U.S. Pat. No. 5,299,491 herein incorporated by reference.

Another method for amplification of nucleic acids is Strand Displacement Amplification (SDA) (Westin et al 2000, Nature Biotechnology, 18, 199-202; Walker et al 1992, Nucleic Acids Research, 20, 7, 1691-1696), which is an isothermal amplification technique based upon the ability of a restriction endonuclease such as HincII or BsoBI to nick the unmodified strand of a hemiphosphorothioate form of its recognition site, and the ability of an exonuclease deficient DNA polymerase such as Klenow exo minus polymerase, or Bst polymerase, to extend the 3′-end at the nick and displace the downstream DNA strand. Exponential amplification results from coupling sense and antisense reactions in which strands displaced from a sense reaction serve as targets for an antisense reaction and vice versa.

Another method for amplification of nucleic acids is Rolling Circle Amplification (RCA) (Lizardi et al. 1998, Nature Genetics, 19:225-232). RCA can be used to amplify single stranded molecules in the form of circles of nucleic acids. In its simplest form, RCA involves the hybridization of a single primer to a circular nucleic acid. Extension of the primer by a DNA polymerase with strand displacement activity results in the production of multiple copies of the circular nucleic acid concatenated into a single DNA strand.

In some embodiments of the invention, RCA is coupled with ligation. For example, a single oligonucleotide can be used both for ligation and as the circular template for RCA. This type of polynucleotide can be referred to as a “padlock probe” or a “RCA probe.” For a padlock probe, both termini of the oligonucleotide contain sequences complementary to a domain within a nucleic acid sequence of interest. The first end of the padlock probe is substantially complementary to a first domain on the nucleic acid sequence of interest, and the second end of the padlock probe is substantially complementary to a second domain, adjacent to the first domain near the first domain. Hybridization of the oligonucleotide to the target nucleic acid results in the formation of a hybridization complex. Ligation of the ends of the padlock probe results in the formation of a modified hybridization complex containing a circular polynucleotide. In some cases, prior to ligation, a polymerase can fill in the gap by extending one end of the padlock probe. The circular polynucleotide thus formed can serve as a template for RCA that, with the addition of a polymerase, results in the formation of an amplified product nucleic acid. The methods of the invention described herein can produce amplified products with defined sequences on both the 5′- and 3′-ends. Such amplified products can be used as padlock probes.

Some aspects of the invention utilize the linear amplification of nucleic acids or polynucleotides. Linear amplification generally refers to a method that involves the formation of one or more copies of the complement of only one strand of a nucleic acid or polynucleotide molecule, usually a nucleic acid or polynucleotide analyte. Thus, the primary difference between linear amplification and exponential amplification is that in the latter process, the product serves as substrate for the formation of more product, whereas in the former process the starting sequence is the substrate for the formation of product but the product of the reaction, i.e. the replication of the starting template, is not a substrate for generation of products. In linear amplification the amount of product formed increases as a linear function of time as opposed to exponential amplification where the amount of product formed is an exponential function of time.

In some embodiments, amplification methods can be solid-phase amplification, polony amplification, colony amplification, emulsion PCR, bead RCA, surface RCA, surface SDA, etc., as will be recognized by one of skill in the art. In some embodiments, amplification methods that results in amplification of free DNA molecules in solution or tethered to a suitable matrix by only one end of the DNA molecule can be used. Methods that rely on bridge PCR, where both PCR primers are attached to a surface (see, e.g., WO 2000/018957 and Adessi et al., Nucleic Acids Research (2000): 28(20): E87) can be used. In some cases the methods of the invention can create a “polymerase colony technology,” or “polony.” referring to a multiplex amplification that maintains spatial clustering of identical amplicons (see Harvard Molecular Technology Group and Lipper Center for Computational Genetics website). These include, for example, in situ polonies (Mitra and Church, Nucleic Acid Research 27, e34, Dec. 15, 1999), in situ rolling circle amplification (RCA) (Lizardi et al., Nature Genetics 19, 225, July 1998), bridge PCR (U.S. Pat. No. 5,641,658), picotiter PCR (Leamon et al., Electrophoresis 24, 3769, November 2003), and emulsion PCR (Dressman et al., PNAS 100, 8817, Jul. 22, 2003). The methods of the invention provide new methods for generating and using polonies.

### Downstream Applications for Amplified Products

An important aspect of the invention is that the methods and compositions disclosed herein can be efficiently, cost-effectively, and with the minimal loss of biological material, utilized for downstream analyses. The amplified and enriched copies of the selected sequence regions of interest are useful for massively parallel sequencing (Next Generation Sequencing methods), multiplexed quantification of large sets of sequence regions of interest, such as by high density qPCR arrays and other highly parallel quantification platforms (selective massively parallel target pre-amplification), as well as generation of libraries with enriched population of sequence regions of interest.

Sequencing

In one embodiment, the invention provides for products ready for amplification in preparation for sequencing.

In some embodiments, the methods are useful for preparing target polynucleotide(s) for sequencing by the sequencing by ligation methods commercialized by Applied Biosystems (e.g., SOLiD sequencing). In general, double stranded fragment polynucleotides can be prepared by the methods of the present invention, and then incorporated into a water-in-oil emulsion along with polystyrene beads and amplified, for example by PCR. In some cases, alternative amplification methods can be employed in the water-in-oil emulsion such as any of the methods provided herein. The amplified product in each water microdroplet formed by the emulsion can interact, bind, or hybridize with the one or more beads present in that microdroplet leading to beads with a plurality of amplified products of substantially one sequence. When the emulsion is broken, the beads float to the top of the sample and are placed onto an array. The methods can include a step of rendering the nucleic acid bound to the beads single-stranded or partially single stranded. Sequencing primers are then added along with a mixture of four different fluorescently labeled oligonucleotide probes. The probes bind specifically to the two bases in the polynucleotide to be sequenced immediately adjacent and 3′ of the sequencing primer to determine which of the four bases are at those positions. After washing and reading the fluorescence signal from the first incorporated probe, a ligase is added. The ligase cleaves the oligonucleotide probe between the fifth and sixth bases, removing the fluorescent dye from the polynucleotide to be sequenced. The whole process is repeated using a different sequence primer until all of the intervening positions in the sequence are imaged. The process allow's the simultaneous reading of millions of DNA fragments in a ‘massively parallel’ manner. This ‘sequence-by-ligation’ technique uses probes that encode for two bases rather than just one allowing error recognition by signal mismatching, leading to increased base determination accuracy.

In some embodiments, the methods are useful for preparing target polynucleotide(s) for sequencing by synthesis using the methods commercialized by 454/Roche Life Sciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; and 7,323,305. In general, double stranded fragment polynucleotides can be prepared by the methods of the present invention, immobilized onto beads, and compartmentalized in a water-in-oil PCR emulsion. In some cases, alternative amplification methods can be employed in the water-in-oil emulsion such as any of the methods provided herein. When the emulsion is broken, amplified fragments remain bound to the beads. The methods can include a step of rendering the nucleic acid bound to the beads single stranded or partially single stranded. The beads can be enriched and loaded into wells of a fiber optic slide so that there is approximately 1 bead in each well. Nucleotides are flowed across and into the wells in a fixed order in the presence of polymerase, sulfhydrolase, and luciferase. Addition of nucleotides complementary to the target strand can result in a chemiluminescent signal that is recorded, such as by a camera. The combination of signal intensity and positional information generated across the plate allows software to determine the DNA sequence.

In some embodiments, the methods are useful for preparing target polynucleotide(s) for sequencing by the methods commercialized by Helicos BioSciences Corporation (Cambridge, Mass.) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058. In general, double stranded fragment polynucleotides can be prepared by the methods of the present invention, and then immobilized onto a flow-cell surface. The methods can include a step of rendering the nucleic acid bound to the flow-cell surface stranded or partially single stranded. Polymerase and labeled nucleotides are then flowed over the immobilized DNA. After fluorescently labeled nucleotides are incorporated into the DNA strands by a DNA polymerase, the surface is illuminated with a laser, and an image is captured and processed to record single molecule incorporation events to produce sequence data.

In some embodiments, the methods are useful for preparing target polynucleotide(s) for sequencing by the sequencing by ligation methods commercialized by Dover Systems. Generally, double stranded fragment polynucleotides can be prepared by the methods of the present invention. The polynucleotides can then be amplified in an emulsion in the presence of magnetic beads. Any amplification methods can be employed in the water-in-oil emulsion such as any of the methods provided herein. The resulting beads with immobilized clonal polynucleotide polonies are then purified by magnetic separation, capped, amine functionalized, and covalently immobilized in a series of flow cells. The methods can include a step of rendering the nucleic acid bound to the flow-cell surface stranded or partially single stranded. Then, a series of anchor primers are flowed through the cell, where they hybridize to the synthetic oligonucleotide sequences at the 3′ or 5′ end of proximal or distal genomic DNA tags. Once an anchor primer is hybridized, a mixture of fully degenerate nonanucleotides ('nonamers') and T4 DNA ligase is flowed into the cell; each of the nonamer mixture's four components is labeled with one of four fluorophores, which correspond to the base type at the query position. The fluorophore-tagged nonamers selectively ligate onto the anchor primer, providing a fluorescent signal that identifies the corresponding base on the genomic DNA tag. Once the probes are ligated, fluorescently labeling the beads, the array is imaged in four colors. Each bead on the array will fluoresce in only one of the four images, indicating whether there is an A, C, G, or T at the position being queried. After imaging, the array of annealed primer-fluorescent probe complex, as well as residual enzyme, are chemically striped using guanidine HCl and sodium hydroxide. After each cycle of base reads at a given position have been completed, and the primer-fluorescent probe complex has been stripped, the anchor primer is replaced, and a new mixture of fluorescently tagged nonamers is introduced, for which the query position is shifted one base further into the genomic DNA tag. Seven bases are queried in this fashion, with the sequence performed from the 5′ end of the proximal tag, followed by six base reads with a different anchor primer from the 3′ end of the proximal tag, for a total of 13 base pair reads for this tag. This sequence is then repeated for the 5′ and 3′ ends of the distal tag, resulting in another 13 base pair reads. The ultimate result is a read length of 26 bases (thirteen from each of the paired tags). However, it is understood that this method is not limited to 26 base read lengths.

In some embodiments, the methods are useful for preparing target polynucleotide(s) from selectively enriched populations of specific sequence regions of interest in a strand-specific manner for sequencing by the methods well known in the art and further described below.

For example the methods are useful for sequencing by the method commercialized by Illumina as described U.S. Pat. Nos. 5,750,341; 6,306,597; and 5,969,119. In general, double stranded fragment polynucleotides can be prepared by the methods of the present invention to produce amplified nucleic acid sequences tagged at one (e.g., (A)/(A′) or both ends (e.g., (A)/(A′) and (C)/(C′)). In some cases, single stranded nucleic acid tagged at one or both ends is amplified by the methods of the present invention (e.g., by SPIA or linear PCR). The resulting nucleic acid is then denatured and the single stranded amplified polynucleotides are randomly attached to the inside surface of flow-cell channels. Unlabeled nucleotides are added to initiate solid-phase bridge amplification to produce dense clusters of double-stranded DNA. To initiate the first base sequencing cycle, four labeled reversible terminators, primers, and DNA polymerase are added. After laser excitation, fluorescence from each cluster on the flow cell is imaged. The identity of the first base for each cluster is then recorded. Cycles of sequencing are performed to determine the fragment sequence one base at a time. For paired-end sequencing, such as for example, when the polynucleotides are labeled at both ends by the methods of the present invention, sequencing templates can be regenerated in-situ so that the opposite end of the fragment can also be sequenced.

In some embodiments, the methods are useful for preparing target polynucleotide(s) for sequencing by the methods commercialized by Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. In general, double stranded fragment polynucleotides can be prepared by the methods of the present invention. The polynucleotides can then be immobilized in zero mode waveguide arrays. The methods may include a step of rendering the nucleic acid bound to the waveguide arrays single stranded or partially single stranded. Polymerase and labeled nucleotides are added in a reaction mixture, and nucleotide incorporations are visualized via fluorescent labels attached to the terminal phosphate groups of the nucleotides. The fluorescent labels are clipped off as part of the nucleotide incorporation. In some cases, circular templates are utilized to enable multiple reads on a single molecule.

Another example of a sequencing technique that can be used in the methods of the provided invention is nanopore sequencing (see e.g. Soni G V and Meller A. (2007) Clin Chem 53: 1996-2001). A nanopore can be a small hole of the order of 1 nanometer in diameter. Immersion of a nanopore in a conducting fluid and application of a potential across it can result in a slight electrical current due to conduction of ions through the nanopore. The amount of current that flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore can represent a reading of the DNA sequence.

Another example of a sequencing technique that can be used in the methods of the provided invention is semiconductor sequencing provided by Ion Torrent (e.g., using the Ion Personal Genome Machine (PGM)). Ion Torrent technology can use a semiconductor chip with multiple layers, e.g., a layer with micro-machined wells, an ion-sensitive layer, and an ion sensor layer. Nucleic acids can be introduced into the wells, e.g., a clonal population of single nucleic can be attached to a single bead, and the bead can be introduced into a well. To initiate sequencing of the nucleic acids on the beads, one type of deoxyribonucleotide (e.g., dATP, dCTP, dGTP, or dTTP) can be introduced into the wells. When one or more nucleotides are incorporated by DNA polymerase, protons (hydrogen ions) are released in the well, which can be detected by the ion sensor. The semiconductor chip can then be washed and the process can be repeated with a different deoxyribonucleotide. A plurality of nucleic acids can be sequenced in the wells of a semiconductor chip. The semiconductor chip can comprise chemical-sensitive field effect transistor (chemFET) arrays to sequence DNA (for example, as described in U.S. Patent Application Publication No. 20090026082). Incorporation of one or more triphosphates into a new nucleic acid strand at the 3′ end of the sequencing primer can be detected by a change in current by a chemFET. An array can have multiple chemFET sensors.

Genetic Analysis

The methods of the present invention can be used in the analysis of genetic information of selective genomic regions of interest as well as genomic regions which may interact with the selective region of interest. Amplification methods as disclosed herein can be used in the devices, kits, and methods known to the art for genetic analysis, such as, but not limited to those found in U.S. Pat. Nos. 6,449,562, 6,287,766, 7,361,468, 7,414,117, 6,225,109, and 6,110,709. In some cases, amplification methods of the present invention can be used to amplify target nucleic acid for DNA hybridization studies to determine the presence or absence of polymorphisms. The polymorphisms, or alleles, can be associated with diseases or conditions such as genetic disease. In other cases the polymorphisms can be associated with susceptibility to diseases or conditions, for example, polymorphisms associated with addiction, degenerative and age related conditions, cancer, and the like. In other cases, the polymorphisms can be associated with beneficial traits such as increased coronary health, or resistance to diseases such as HIV or malaria, or resistance to degenerative diseases such as osteoporosis, Alzheimer's or dementia.

Additional Uses of the Methods of the Invention:

The methods of the invention can also find use in the analysis of genomic DNA modifications such as methylation. It is envisioned that directed hybridization of multiplicity of oligonucleotides useful for carrying out the methods of the invention can be designed to specifically hybridize to regions of interest and the action of the tethered NA-modifying enzymes can specifically act, or be prevented from acting on modified nucleotides in the complex genomic DNA sample, as compared to the non modified canonical nucleotides. It is also envisioned that the selected tethered NA-modifying enzyme can specifically recognize nucleotide modifications and mark the sequence for subsequent cleavage.

The tethered NA-modifying enzyme can also act on sequence regions in the complex genomic DNA as these come in contact, are come to close proximity, to the selected sequence regions of interest.

### Kits

Any of the compositions described herein may be comprised in a kit. In a non-limiting example the kit, in suitable container means, comprises: one oligonucleotide or a plurality of oligonucleotides, each coupled with at least one ligand, and a ligand-binding agent coupled to a NA-modifying enzyme. The kit can further contain adapters and/or reagents useful for ligation. The kit can further optionally contain a DNA-polymerase. The kit can further optionally contain reagents for amplification, for example reagents useful for single primer isothermal amplification methods. The kit can further optionally contain reagents for sequencing, for example, reagents useful for next-generation massively parallel sequencing methods.

The containers of the kits can generally include at least one vial, test tube, flask, bottle, syringe or other containers, into which a component may be placed, and preferably, suitably aliquoted. Where there is more than one component in the kit, the kit also can generally contain a second, third or other additional container into which the additional components can be separately placed. However, various combinations of components can be comprised in a container.

When the components of the kit are provided in one or more liquid solutions, the liquid solution can be an aqueous solution. However, the components of the kit can be provided as dried powder(s). When reagents and/or components are provided as a dry powder, the powder can be reconstituted by the addition of a suitable solvent.

A kit can include instructions for employing the kit components as well the use of any other reagent not included in the kit. Instructions can include variations that can be implemented.

### EXAMPLES

### Example 1

### Method 1, Sequence-Specific Enrichment of a Target Region of Interest from Denatured Double-Stranded Genomic DNA

Genomic DNA sample is mixed with a solution comprising oligonucleotides designed to specifically hybridize to the sequence regions of interest to be enriched and amplified. The oligonucleotides of the invention comprise a label linked to one end, preferably the 5′-end. The label can be any member of a specific binding pair, for example biotin. The ligand (label) can be attached to the end of the oligonucleotides by a linker. The length and composition of the linker will depend on the desired flexibility. Various linkers and methods for synthesis are well known in the art. The length and composition (% GC) of the oligonucleotide are selected to provide efficient hybridization to the multiplicity of sequence regions of interest at a predetermined stringency. The design requirements are well known in the art. Methods used for the design and selection of oligonucleotides employed for analyses employing high density microarrays are well known in the art and are suitable for the design of the multiplicity of oligonucleotides suitable for use in the methods of the current invention. The multiplicity of oligonucleotides for use in a method of the invention can be synthesized by any of the known methods, such as provided by synthesis employing microarrays as provided by various venders including Agilent, LC Sciences, Febit and the like. The number of oligonucleotides selected for the enrichment of a given sequence region of interest can vary from a single oligonucleotide to numerous oligonucleotides dependent on the length of the sequence region of interest.

The mixture is heated to denature the double stranded genomic DNA (dsDNA) and is cooled to allow hybridization of the multiplicity of oligonucleotides comprising a label or ligand to the specific sequence regions of interest (FIG. 1).

The hybridized oligonucleotides are optionally extended along the sequence region of interest by DNA polymerase.

An example of the process is further illustrated in FIG. 2. A mixture comprising nucleic acid modifying enzyme conjugated to a second member of the specific binding pair, for example avidin or streptavidin when the label or ligand is biotin, is added to the reaction mixture. Binding of the second member of the specific binding pair to the ligand attached to the 5′-end of the oligonucleotide results in anchoring the NA-modifying enzyme to the hybridized oligonucleotide or the extended oligonucleotide.

The NA-modifying enzyme is selected for specificity for dsDNA. The enzyme may be a duplex-specific endonuclease or a blunt end frequent cutter restriction enzyme, or other restriction enzymes. Other NA modification enzymes are also envisioned to be useful for carrying out the methods of the invention.

The NA-modifying enzyme employed may require interaction with solution phase subunit. Various duplex specific restriction enzymes require dimer formation for cleavage of both strands of the duplex. The required dimerization (e.g., the interaction of the tethered enzyme and a solution phase enzyme) will increase the specificity of cleavage by limiting it to the site of the sequence regions of interest.

The cleavage of both strands of the duplex formed by the hybridized (and optionally extended) oligonucleotide and the sequence region of interest results in the formation of a double stranded product (may be a partial double stranded product) without the tethered NA modification enzyme, and a partial double stranded product with the tethered NA-modifying enzyme (FIG. 3). The latter comprises a short segment of the oligonucleotide which may be too short for stable hybridization, thus preventing ligation of the adapters to that product. Alternatively, adapter ligation to the desired end is controlled by polarity and sequence content of the ligation ends. The double stranded, or partially dsDNA products, without the label or ligand bearing segment of the oligonucleotide, are substrates for ligation.

Various adapter designs are envisioned. Some examples are shown in FIGS. 4, 5, 6 and 7. The adapters are designed to comprise a double-stranded portion which can be ligated to the dsDNA (or dsDNA with overhang) products. Various ligation process and reagents are known in the art and are useful for carrying out the methods of the invention. For example blunt ligation can be employed. Similarly, a single dA nucleotide can be added to the 3′-end of the dsDNA product, by a polymerase lacking 3′-exonuclease, and the adapter designed to comprise a dT overhang (or the reverse). This design allows hybridization of the two components to be subsequently ligated. Other ligation strategies and the corresponding reagents are known in the art, and kits and reagents for carrying out efficient ligation reactions are commercially available.

A common feature of the adapter designs depicted in FIGS. 4 to 7 is the 3′-single stranded DNA comprising a unique sequence which is hybridizable to an amplification primer employed in subsequent amplification step. As shown in the above mentioned figures, strand specific amplification of the sequence regions of interest is carried out by the single primer isothermal amplification (SPIA). A chimeric RNA-DNA amplification primer, DNA polymerase with strand displacement activity, and RNase H are employed in the isothermal linear amplification (see e.g., U.S. Pat. Nos. 7,402,386, 7,354,717, 7,351,557, 7,771,946, and 7,771,934). The single amplification primer comprises a 3′-DNA sequence and a 5′-RNA sequence. The primer sequence is hybridizable to the 3′-ssDNA portion of the ligation product, and can be partially hybridizable to the sequence of the dsDNA portion of the adapter. The dsDNA portion of the adapters can further comprise indexing or bar-coding sequences designed to mark either the samples or sequences of interests. The ability to incorporate marking and/or bar-coding information in the adapter design further expands the high multiplexing ability of the methods of the invention.

A novel stem-loop adapter design is shown in FIG. 7. The stem portion of the adapter comprises an RNA-DNA heteroduplex formed by hybridization of the 3′-portion of the oligonucleotide, comprising RNA sequence, to its 5′-end portion. The 3′-end of the oligonucleotide can comprise a dNTP to facilitate ligation, when required for efficient ligation using the ligase of choice. The stem may or may not be blunt ended, as required for efficient hybridization to the cleavage products of the sequence sequences of interest generated by the methods of the invention. The ligation products comprise a loop at one end attached to the cleavage product via an RNA-DNA heteroduplex. The RNA portion of the heteroduplex is susceptible to cleavage by RNase H. The adapter further comprises a loop sequence that is hybridizable to the amplification primer used for the single primer amplification of the sequence regions of interest. Cleavage of the RNA portion of the stem by RNase H results in the formation of a ssDNA 3′-overhang which is the primer binding site. Amplification of the strand specific sequence region of interest can be carried out by repeated primer extension steps or preferably by the isothermal single primer linear amplification (SPIA). Following ligation, the ligation product is added to amplification reaction mixture comprising the chimeric amplification primer, RNase H and DNA polymerase with strand displacement activity. The RNA portion of the stem heteroduplex is cleaved by RNase H to open the stem loop. Multiple copies of the strand specific selected sequence regions of interest is carried out by hybridization of the chimeric RNA-DNA chimeric primer, extension of the primer along the target strand by DNA polymerase, cleavage of the RNA portion of the hybridized primer by RNase H, and hybridization of a new primer at the free primer binding site. Repeated cycles of this process generate multiple copies of the ligated target strand.

The entire population of ligation products, comprising the multiplicity of selected sequence regions of interest, is amplified using the generic chimeric amplification primer, thus generating multiple copies of the strand-specific selected sequence regions of interest. All copies comprise the copies of the targeted sequence regions of interest and can further comprise the stem sequence of the adapter which in turn can comprise marking and/or bar coding information.

The products of the selected enrichment and amplification are useful for quantification of the various targeted sequence regions of interest in the complex genomic nucleic acid sample, for example by qPCR. Massively parallel quantification platforms have been developed and can be useful for genome-wide quantification of sequences of interest. The products can further be useful for generation of libraries of sequence regions of interest which can be analyzed by sequencing with or without prior cloning. The products can be rendered suitable for massively parallel sequencing using any of various methods and platforms developed in recent years. In addition, the marked and bar coded products generated from various samples (e.g., individually) can be combined and analyzed in a mixture. Comparative analysis of the libraries obtained from various samples can be used for the elucidation of the effect of sequence, structural or copy number variations on phenotype, development, differentiation and the like.

### Example 2

### Method 2, Sequence-Specific Enrichment of a Target Region of Interest from Non-Denatured Double-Stranded Genomic DNA

Targeted tethering of a NA-modifying enzyme can be achieved by sequence-specific triplex formation as shown in FIGS. 8 and 9. Oligonucleotides comprising a label or ligand are designed to specifically hybridize to dsDNA sequence regions of interest in a complex genomic DNA sample to form triplex. The targeted tethering of the NA-modifying enzyme is carried out in a manner similar to that of Method 1. The modifying enzyme is selected to directly or indirectly cleave the dsDNA target upstream of the triplex, and the targeted cleavage site is rendered suitable for ligation of adapters as described for Method 1. The generation of enriched and amplified copies of the multiplicity of sequence regions of interest is carried out similar to that described above (Method 1).

The method is useful for generation of copies of strand specific sequence regions of interest from complex nucleic acid without the need to denature the dsDNA input DNA, thus enabling enrichment and analysis of a multiplicity of sequence regions of interest in the native complex nucleic acid sample. It is envisioned that the method will find use for studies and analyses carried out in situ, enable studies and analysis of complex genomic DNA in single cells or collection of very small well defined cell population, as well as the analysis of complex genomic DNA without disruption of chromatin structures.

### Example 3

### Use of Sequence-Specific Enrichment of a Target Region of Interest from Denatured Double-Stranded Genomic DNA for Diagnosing Cancer

Genomic DNA is isolated from a patient sample (e.g., a tumor sample) and mixed with oligonucleotides that comprise a biotin linked to the 5′ end through a 12 carbon spacer linker. The genomic DNA is denatured, and the biotin linked oligonucleotides are hybridized to the genomic DNA and extended with a DNA polymerase. A “blunt-cutter” endonuclease conjugated to streptavidin is added to the reaction mixture, and the endonuclease binds the biotin linked oligonucleotides via the conjugated streptavidin. The endonuclease cleaves both strands of the duplex formed by the hybridized and extended oligonucleotide. Adapters are added to the end of cleaved DNA. The adaptors permit binding of chimeric primers for subsequent linear amplification of the cleaved DNA. The adaptors also provide binding sites for primers that permit Next Generation Sequencing (e.g., Illumina sequencing). The results of the sequencing are analyzed to determine the presence of absence of mutations associated with cancer.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for selectively partitioning a plurality of target regions of interest from complex DNA comprising:
a. hybridizing one or more oligonucleotides to the target regions of interest to form a plurality of target-oligonucleotide complexes;
b. tethering a nucleic acid-modifying enzyme to a target-oligonucleotide complex via the oligonucleotide; and
c. cleaving the target region of interest in the complex, thereby releasing the target region of interest from the enzyme.

2. The method of claim 1 wherein the one or more oligonucleotides hybridized to the target regions of interest are extended along the target region of interest by DNA polymerase prior to step b.

3. The method of claim 1 wherein the complex DNA comprises double-stranded DNA.

4. The method of claim 1 wherein the complex DNA comprises genomic DNA.

5. The method of claim 4 wherein the genomic DNA comprises a mixture of genomic DNA from more than one organism.

6. The method of claim 1 wherein the complex DNA comprises cDNA.

7. The method of claim 6 wherein the cDNA is generated from a mixture of DNAs from more than one organism.

8. The method of claim 1 wherein the selective partitioning is strand-specific.

9. The method of claim 1 wherein the enzyme is a DNA duplex-specific endonuclease.

10. The method of claim 1 wherein the enzyme is a restriction enzyme.

11. The method of claim 1 wherein the method further comprises denaturing the complex DNA prior to hybridization of the oligonucleotides.

12. The method of claim 1 wherein the method further comprises the formation of partial triplexes.

13. The method of claim 1 wherein the method further comprises ligating adapters to the target regions of interest once released from the enzyme.

14. The method of claim 13 wherein the adapter is selected from a group consisting of a double stranded adapter with an overhang at one end, a double stranded adapter with a 3′ single stranded overhang, an adapter that comprises a RNA-DNA heteroduplex, an adapter that comprises a chimeric DNA-RNA oligonucleotide and a stem-loop adapter.

15. The method of claim 1 wherein the selective partitioning is carried out directly on the complex DNA.

16. The method of claim 1 wherein said method does not involve amplifying the complex DNA prior to selective partitioning.

17. The method of claim 1 wherein the method further comprises amplifying the partitioned target regions of interest thereby enriching for the target regions of interest.

18. The method of claim 17 wherein the amplifying comprises single primer isothermal amplification.

19. The method of claim 17 wherein the method further comprises sequencing of the amplified products.

20. The method of claim 19 wherein the sequencing is performed using a massively parallel sequencing method.

21. The method of claim 1 wherein the enzyme is synthetic, semisynthetic, or recombinant.

22. The method of claim 1 wherein the ligand is selected from the group consisting of a small molecule, an antigen, an antibody, hybrid antibody or antibody fragment, an siRNA, an antisense RNA, an aptamer, a peptide and a first member of a specific binding pair.

23. The method of claim 1 wherein the ligand-binding component is selected from the group consisting of a small molecule, an antigen, an antibody, hybrid antibody or antibody fragment, an siRNA, an antisense RNA, an aptamer, a protein therapeutic, or a peptide, a ligand binding protein, and a second member of specific binding pair.

24. The method of claim 1 wherein at least 100 different regions of interest are selectively partitioned.

25. The method of claim 1 wherein at least 100 oligonucleotides are used for selectively partitioning the target region of interest from complex DNA.

26. The method of claim 25 wherein each oligonucleotide is coupled to the same ligand.

27. The method of claim 1 wherein the ligand is biotin and the ligand-binding component is avidin or streptavidin.

28. A method of preparing amplification-ready selectively targeted regions of interest from double-stranded DNA wherein the method comprises:
a. denaturing the double-stranded DNA thereby generating single-stranded DNA;
b. hybridizing to the single-stranded target regions of interest one or more oligonucleotides to form partial duplexes, each oligonucleotide being coupled to a ligand;
c. contacting the ligands with a ligand-binding component coupled to a nucleic acid-modifying enzyme, wherein the enzyme cleaves the target regions of interest thereby obtaining products comprising the target regions of interest free of the enzyme; and
d. ligating adapters to the products, whereby the targeted regions of interest are amplification-ready.

29. A method of preparing amplification-ready selectively targeted regions of interest from double-stranded DNA wherein the method comprises:
a. hybridizing to the double-stranded target regions of interest one or more oligonucleotides to form partial triplexes, each oligonucleotide being coupled to a ligand;
b. contacting the ligands with a ligand-binding component coupled to a nucleic acid-modifying enzyme, wherein the enzyme cleaves the target regions of interest thereby obtaining products comprising the target regions of interest free of the enzyme; and
c. ligating adapters to the products, whereby the targeted regions of interest are amplification-ready.

30. The method of claim 28 or 29 wherein the double-stranded DNA comprises genomic DNA.

31. The method of claim 28 or 29 wherein the double-stranded DNA comprises cDNA.

32. The method of claim 31 wherein the cDNA is generated from a mixture of DNAs from more than one organism.

33. The method of claim 30 wherein the genomic DNA comprises a mixture of genomic DNA from more than one organism.

34. The method of claim 28 or 29 wherein the method is strand-specific.

35. The method of claim 28 or 29 wherein the enzyme is a DNA-duplex specific endonuclease.

36. The method of claim 28 or 29 wherein the enzyme is a restriction enzyme.

37. The method of claim 28 or 29 wherein the adapter is selected from a group consisting of a double stranded adapter with an overhang at one end, a double stranded adapter with a 3′ single stranded overhang, an adapter that comprises a RNA-DNA heteroduplex, an adapter that comprises a chimeric DNA-RNA oligonucleotide and a stem-loop adapter.

38. The method of claim 28 or 29 wherein the method is carried out directly on the double-stranded DNA.

39. The method of claim 28 wherein said method does not involve amplifying the double-stranded DNA until after step d.

40. The method of claim 29 wherein said method does not involve amplifying the double-stranded DNA until after step c.

41. The method of claim 28 or 29 wherein the method further comprises amplifying the target regions of interest, thereby enriching for the target sequence regions of interest.

42. The method of claim 41 wherein the amplifying comprises single primer isothermal amplification.

43. The method of claim 41 wherein the method further comprises sequencing of the amplified products.

44. The method of claim 43 wherein the sequencing is performed using a massively parallel sequencing method.

45. The method of claim 28 or 29 wherein the enzyme is synthetic, semisynthetic or recombinant.

46. The method of claim 28 or 29 wherein the ligand is selected from the group consisting of a small molecule, an antigen, an antibody, hybrid antibody or antibody fragment, an siRNA, an antisense RNA, an aptamer, a peptide and a first member of a specific binding pair.

47. The method of claim 28 or 29 wherein the ligand-binding component is selected from the group consisting of a small molecule, an antigen, an antibody, hybrid antibody or antibody fragment, an siRNA, an antisense RNA, an aptamer, a protein therapeutic, or a peptide, a ligand binding protein, and a second member of specific binding pair.

48. The method of claim 28 or 29 wherein the ligand is biotin and the ligand-binding component is avidin or streptavidin.

49. The method of claim 28 or 29 wherein at least 100 different regions of interest are prepared for amplification.

50. The method of claim 28 or 29 wherein at least 100 oligonucleotides are used for preparing the target regions of interest for amplification.

51. The method of claim 28 or 29 wherein each oligonucleotide is coupled to the same ligand.

52. A DNA complex comprising:
a. DNA comprising at least one DNA strand;
b. an oligonucleotide hybridized to the DNA wherein the oligonucleotide is coupled to a ligand; and
c. a ligand-binding component coupled to a nucleic acid-modifying enzyme, wherein the ligand and the ligand-biding component are further coupled to each other.

53. The complex of claim 52 wherein the DNA comprises genomic DNA.

54. The complex of claim 52 wherein the DNA is double stranded and the complex is a partial triplex.

55. The complex of claim 52 wherein the DNA is single stranded and the complex is a partial duplex.

56. The complex of claim 52 wherein the nucleic acid-modifying enzyme is synthetic, semisynthetic, or recombinant.

57. The complex of claim 52 wherein the ligand is selected from the group consisting of a small molecule, an antigen, an antibody, hybrid antibody or antibody fragment, an siRNA, an antisense RNA, an aptamer, a peptide and a first member of a specific binding pair.

58. The complex of claim 52 wherein the ligand binding component is selected from the group consisting of a small molecule, an antigen, an antibody, hybrid antibody or antibody fragment, an siRNA, an antisense RNA, an aptamer, a protein therapeutic, or a peptide, a ligand binding protein, and a second member of specific binding pair.

59. The complex of claim 52 wherein the ligand is biotin and the ligand binding component is avidin or streptavidin.

60. The complex of claim 52 wherein the oligonucleotide is further extended with a DNA polymerase.

61. A kit comprising:
a. one or more oligonucleotides, each coupled to a ligand; and
b. a ligand-binding component that selectively binds to said ligand, wherein said ligand-binding component is coupled to a nucleic acid-modifying enzyme.

62. The kit of claim 61 wherein the kit further comprises reagents for amplification.

63. The kit of claim 62 wherein the kit further comprises an adapter for amplification.

64. The kit of claim 63 wherein the adapter is selected from a group consisting of a double stranded adapter with an overhang at one end, a double stranded adapter with a 3′ single stranded overhang, an adapter that comprises a RNA-DNA heteroduplex, an adapter that comprises a chimeric DNA-RNA oligonucleotide and a stem-loop adapter.

65. The kit of claim 62 wherein the kit further comprises reagents for sequencing.

66. The kit of claim 65 wherein the sequencing reagents comprise reagents for a massively parallel sequencing method.

67. The kit of claim 62 wherein the reagents are reagents for performing single primer isothermal amplification.

68. The kit of claim 61 wherein the kit further comprises a DNA polymerase.

69. The kit of claim 61 wherein the kit further comprises a plurality of oligonucleotides.

70. The kit of claim 69 wherein the plurality of oligonucleotides are coupled to different ligands.

71. The kit of claim 61 wherein the ligand is selected from the group consisting of a small molecule, an antigen, an antibody, hybrid antibody or antibody fragment, an siRNA, an antisense RNA, an aptamer, a peptide and a first member of a specific binding pair.

72. The kit of claim 61 wherein the ligand binding component is selected from the group consisting of a small molecule, an antigen, an antibody, hybrid antibody or antibody fragment, an siRNA, an antisense RNA, an aptamer, a protein therapeutic, or a peptide, a ligand binding protein, and a second member of specific binding pair.

73. The kit of claim 61 wherein the ligand is biotin and the ligand-binding component is avidin or streptavidin.

74. The kit of claim 61 wherein the nucleic acid modifying enzyme is synthetic, semisynthetic, or recombinant.

75. The kit of claim 61 wherein the nucleic acid modifying enzyme is a DNA duplex-specific endonuclease.

76. The kit of claim 61 wherein the nucleic acid modifying enzyme is a restriction endonuclease.

77. A plurality of amplification-ready partial DNA duplexes generated from genomic DNA, each duplex comprising at least one DNA strand comprising the sequence of a target region of interest wherein the duplex is further ligated to an adaptor for single primer isothermal amplification.
